(19) United States
(12) Patent Application Publication
(10) Pub. No.: US 2004/0138212 A1
(43) Pub. Date: Jul. 15, 2004

(54) Disubstituted 1,7-guanines

(76) Inventors: Alberto Bargiotti, Milan (IT); Antonella Ermoli, Buccinasco (IT); Maria Menichincheri, Milan (IT); Ermes Vanotti, Milan (IT); Luisella Bonomini, Cesano Maderno (IT); Antonella Fretta, Corsico (IT)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/476,343

(22) PCT Filed: Jun. 8, 2001
(86) PCT No.: PCT/EP01/06624

(30) Foreign Application Priority Data
May 4, 2001 (GB) 0111071.7

(51) Int. Cl. A61K 31/522; C07D 473/18

(52) U.S. Cl. 514/225.2; 514/263.2; 514/263.21; 514/263.22; 514/263.37; 544/276; 544/43; 544/268

(57) ABSTRACT

The present invention relates to 1,7 disubstituted guanines inhibitors possessing antitumor activity and to a process for preparing the same. Furthermore, these compounds are expected to enhance the efficacy of other chemotherapeutic agents, in the treatment of cancer.

### FIELD OF THE INVENTION

The present invention relates to compounds that inhibit telomerase activity, to a process for their preparation, to their use as medicaments and to pharmaceutical compositions comprising them.

### BACKGROUND OF THE INVENTION

Cancer is one of the major causes of disease and the second leading cause of death in the western world. Most cancer patients still die due to metastatic disease. Despite the great increase in the knowledge and understanding of the regulatory mechanisms involved in the onset of malignancy, currently available treatments (including surgery, radiation and a variety of cytoreductive and hormone-based drugs, used alone or in combination, are still highly non specific and toxic to the patient, causing severe side effects including nausea and vomiting, hair loss, diarrhea, fatigue and ulcerations. These problems evidence the need for new and more effective anti-cancer therapies.

Recently an understanding of the mechanisms by which normal cells reach the state of replicative senescence, i.e. the loss of proliferative capacity that cells normally undergo in the cellular aging process has begun to emerge and in this respect telomerase appears to have a central role.

Telomerase is a ribonucleoprotein enzyme responsible in most eukaryotes for the complete replication and maintenance of chromosome ends, or telomeres, which are composed of repeated DNA sequences (in particular human telomeres are formed by 5′-TTAGGG repeats). Telomerase binds to telomeric DNA using as a template a sequence contained within the RNA component of the enzyme necessary for the addition of the short sequence repeats to the chromosome 3′ end (see Blackburn 1992, Annu. Rev. Biochem., 61, 113-129). In most human somatic cells telomerase activity cannot be detected and telomeres shorten with successive cell division: in fact actively dividing normal cells have the potential to lose 50-200 base pairs after each round of cell division, resulting in shortening of telomeres. Recently it has been hypothesized that the cumulative loss of telomeric DNA over repeated cell divisions may act as a trigger for cellular senescence and aging, and that regulation of telomerase may have important biological implications (see Harley 1991, Mutation Research, 256, 271-282). In fact in the absence of telomerase, telomeres shortening will eventually lead to cellular senescence by various mechanisms. This phenomenon, thought to be responsible for cellular aging, is termed the “mitotic clock” (see Holt et al. Nat. Biotechnol., 1996, 15, 1734-1741).

Telomerase activity is restored in immortalised cell lines and in more than 85% of human tumors, thus maintaining telomeres length stable (see Shay, J. W. and Bacchetti, S. Eur. J. Cancer, 1997, 33, 787-791). Thus in cancer cells having telomerase activity and where the malignant phenotype is due to the loss of cell cycle or growth controls or other genetic damage, telomeric DNA is not lost during cell division and telomers are maintained, thereby allowing the cancer cells to become immortal, leading to a terminal prognosis for the patient.

Telomerase inhibition can lead to telomere shortening in tumors and subsequent senescent phenotype (see Feng et al. Science, 1995, 269, 1236-1241). Moreover it has been recently shown (Hahn et al. Nature Med., 1999, 5, 1164-1170) that inhibition of telomerase activity by expressing in tumor cells a catalytically-inactive form of human TERT (TElomerase Reverse Transcriptase, the catalytic subunit of the enzyme) can cause telomere shortening and arrest of cell growth and apoptosis. In addition peptide-nucleic acids and 2′-O-MeRNA oligomers complementary to the template region of the RNA component of the enzyme have been reported to cause inhibition of telomerase activity, telomere shortening and cell death in certain tumor cell lines (see Herbert et al. PNAS, 1999, 96, 14276-14281; Shammas et al. Oncogene, 1999, 18, 6191-6200). These data support inhibition of telomerase activity as an innovative, selective and useful method for the development of new anticancer agents.

Thus compounds that inhibit telomerase activity can be used to treat cancer, as cancer cells express telomerase activity, while normal human somatic cells usually do not express telomerase activity at biologically relevant levels (i.e., at levels sufficient to maintain telomere length over many cell divisions). Also telomere length in tumors is reduced compared with non-transformed cells giving the possibility of a therapeutic window (see Nakamura et al. Cancer Letters 158, 2000, 179-184). Therefore a need exists to find molecules that inhibit the activity of telomerase and interfere with the growth of many types of cancer.

The present invention fulfills such a need by providing a highly general method of treating many—if not most—malignancies, as demonstrated by the highly varied human tumor cell lines and tumors having telomerase activity. Since the compounds of the present invention can be effective in providing treatments that discriminate between malignant and normal cells to a high degree, avoiding many of the deleterious side-effects present with most current chemotherapeutic regimes which rely on agents that kill dividing cells indiscriminately, they are also expected to exhibit greater safety and lack of toxic effects in comparison with traditional chemotherapeutic anticancer agents.

### SUMMARY OF THE INVENTION

The present invention relates to novel purinic derivatives active as telomerase inhibitors, to their use as therapeutic agents, in particular as antitumoral agents, to a process for their preparation and to pharmaceutical compositions comprising them. These and other aspects of the invention are described in greater detail below.

### DETAILED DESCRIPTION OF THE INVENTION

Object of the present invention are 1,7 disubstituted purines of formula (I)

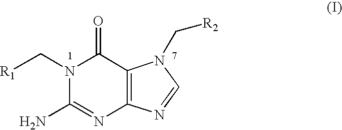

wherein

R1 and R2 represent each independently:

a) phenyl substituted by from 1 to 3 substituents chosen from halogen, cyano, nitro, amino, carboxyl, (C₁-C₆ alkoxy)carbonyl, phenyl and phenoxy;

b) a fused bicycle carbocyclic residue chosen from 1-naphthyl and 2-naphthyl;

c) imidazo[1,2-a]pyridin-2-yl optionally substituted with halogen;

d) a fused tricycle residue chosen from anthraquinonyl, phenothiazinyl, acridinyl and fluorenyl;

e) a fused benzoheterocycle residue chosen from benzodioxolyl, benzoxadiazolyl, quinolinyl, isoquinolinyl and quinazolinyl; or

f) a phenylheterocycle residue chosen from phenylimidazolyl, phenylpyrazolyl, phenyloxadiazolyl, phenylpyrrolyl, phenylfuranyl, phenylthiadiazolyl, phenyloxazolyl, phenylthiazolyl and phenyltetrazolyl;

and the pharmaceutically acceptable salts thereof.

The compounds described herein contain a ketonic carbonyl group as part of a heterocyclic ring system. Such carbonyl group may exist in part or principally in the “keto” form and in part or principally as “enol”, form of the ketone group present. Compounds of the present invention are meant to include both “keto” and “enol” tautomeric forms.

The compounds described herein contain an “imine” or “enamine” group. Such group may exist in part or principally in the “imine” form and in part or principally in the “enamine” form. Compounds of the present invention having said “imine” or “enamine” group are meant to include both “imine” and “enamine” tautomeric forms. It is therefore understood that the present invention includes in its scope the possible tautomeric form of the compounds of formula (I) represented by the following tautomeric formulae (Ia) and (Ib):

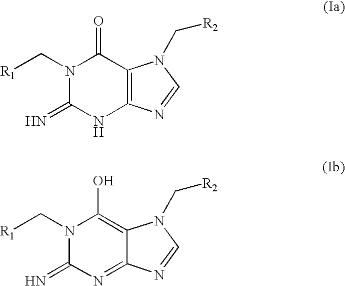

wherein R₁ and R₂ are as defined above. Accordingly, the chemical compounds provided by the present invention are named throughout the description of the invention according to the chemical nomenclature provided for the compounds of formula (I), (Ia) and (Ib) on the basis of the structural evidence validated by people skilled in the art. The compounds of formula (I), (Ia) and (Ib) are herein defined as the “compounds of the present invention”, the “compounds of the invention” and/or the “active principles of the pharmaceutical compositions of the invention”.

The present invention also includes within its scope pharmaceutically acceptable bio-precursors (otherwise known as pro-drugs) of the compounds of formula (I), i.e. compounds which have a different formula (I) above, but which nevertheless upon administration to a human being are converted directly or indirectly in vivo into a compound of formula (I).

A halogen atom is chlorine, bromine, iodine or fluorine, preferably it is chlorine or fluorine.

A (C₁-C₆ alkoxy)carbonyl group is, preferably, a (C₁-C₄ alkoxy)carbonyl group, for example, methoxycarbonyl, ethoxycarbonyl, propoxycarbonyl or butoxycarbonyl.

Pharmaceutically acceptable salts of the compounds of the invention include acid addition salts, with inorganic, e.g. nitric, hydrochloric, hydrobromic, sulphuric, perchloric and phosphoric acids, or organic, e.g. acetic, propionic, glycolic, lactic, oxalic, malonic, malic, maleic, tartaric, citric, benzoic, cinnamic, mandelic and salicylic acids, and salts with inorganic, e.g. alkali metal, especially sodium or potassium bases, or alkaline-earth metal, especially calcium or magnesium bases, or with organic bases, e.g. alkylamines, preferably triethyl-amine.

Preferred compounds of the invention are compounds of formula (I) as defined above, wherein R₁ and R₂ represent, each independently, phenyl substituted by from 1 to 3 substituents chosen from halogen, cyano, nitro, amino, carboxyl, (C₁-C₆ alkoxy)carbonyl, phenyl and phenoxy; 2-naphthyl; imidazo[1,2-a]pyridin-2-yl optionally substituted with chloro; anthraquinonyl; a fused benzoheterocyclicle residue chosen from benzodioxolyl, benzoxadiazolyl and quinolinyl; or phenylimidazolyl; and the pharmaceutically acceptable salts thereof.

Examples of preferred compounds of the invention are:

2-amino-1,7-bis(2-naphthylmethyl)-1,7-dihydro-6H-purin-6-one (compound 1);

2-amino-1-(3,4-dichlorobenzyl)-7-(2-naphthylmethyl)-1,7-dihydro-6H-purin-6-one (compound 2);

2-amino-7-(2-naphthylmethyl)-1-(3-phenoxybenzyl)-1,7-dihydro-6H-purin-6-one (compound 3);

2-amino-7-(2-naphthylmethyl)-1-(2-quinolinylmethyl)-1,7-dihydro-6H-purin-6-one (compound 4);

2-amino-1-(2,1,3-benzoxadiazol-5-ylmethyl)-7-(2-naphthylmethyl)-1,7-dihydro-6H-purin-6-one (compound 5);

2-amino-1-(1,3-benzodioxol-5-ylmethyl)-7-(2-naphthylmethyl)-1,7-dihydro-6H-purin-6-one (compound 6);

2-amino-7-(2-naphthylmethyl)-1-(4-nitrobenzyl)-1,7-dihydro-6H-purin-6-one (compound 7);

2-amino-1-(3,4-difluorobenzyl)-7-(2-naphthylmethyl)-1,7-dihydro-6H-purin-6-one (compound 8);

4-{[2-amino-7-(2-naphthylmethyl)-6-oxo-6,7-dihydro-1H-purin-1-yl]methyl}benzonitrile (compound 9);

2-amino-1-[4-(1H-imidazol-1-yl)benzyl]-7-(2-naphthylmethyl)-1,7-dihydro-6H-purin-6-one (compound 10);

2-{[2-amino-7-(2-naphthylmethyl)-6-oxo-6,7-dihydro-1H-purin-1-yl]methyl}anthra-9,10-quinone (compound 11);

methyl 4-{[2-amino-7-(2-naphthylmethyl)-6-oxo-6,7-dihydro-1H-purin-1-yl]methyl}benzoate (compound 12);

methyl 4-({2-amino-7-[4-(methoxycarbonyl)benzyl]-6-oxo-6,7-dihydro-1H-purin-1-yl}methyl) benzoate (compound 13);

methyl 3-{[2-amino-7-(2-naphthylmethyl)-6-oxo-6,7-dihydro-1H-purin-1-yl]methyl}benzoate (compound 14);

methyl 2-{[2-amino-7-(2-naphthylmethyl)-6-oxo-6,7-dihydro-1H-purin-1-yl]methyl}benzoate (compound 15);

methyl 4-{[2-amino-1-(2-naphthylmethyl)-6-oxo-1,6-dihydro-7H-purin-7-yl]methyl}benzoate (compound 16);

2-amino-1-[(6-chloroimidazo[1,2-a]pyridin-2-yl)methyl]-7-(2-naphthylmethyl)-1,7-dihydro-6H-purin-6-one (compound 17);

and the pharmaceutically acceptable salts thereof.

It is another object of the present invention a method for inhibiting telomerase enzyme, which comprises contacting said enzyme with an effective amount of a compound having the above formula(I) or a pharmaceutically acceptable salt thereof.

It is a further object of the present invention a method for treating a telomerase-modulated disease, which comprises administering to a mammal a therapeutic effective amount of a compound having the above formula (I) or a pharmaceutically acceptable salt thereof.

It is a still further object of the present invention a method for treating a cancer disease related to a deranged cancer cell growth mediated by telomerase enzyme activity, which comprises administering to a mammal a therapeutic effective amount of a compound having the above formula (I) or a pharmaceutically acceptable salt thereof.

It is still another object of the present invention a method for treating a cancer, which comprises administering to a mammal a therapeutic effective amount of a compound having the above formula (I) or a pharmaceutically acceptable salt thereof.

According to still another aspect of the invention, a method is provided which involves the use of a compound having the above formula (I) or a pharmaceutically acceptable salt thereof in the preparation of a medicament. In particular embodiments, the medicament is for treating a proliferative disorder (e.g. a cancer). The present invention therefore also provides a compound having the above formula (I) or a pharmaceutically acceptable salt thereof for use in the preparation of a medicament having anticancer activity.

A still further object of the present invention is to provide a pharmaceutical composition comprising a pharmaceutically acceptable carrier and/or diluent and, as an active principle, a compound of formula (I) as defined above or a pharmaceutically acceptable salt thereof.

The present invention also provides the use of a compound of formula (I) as defined above or a pharmaceutically acceptable salt thereof, in the preparation of a medicament for use as antitumor agent.

According to the present invention, 1,7 disubstituted guanines of formula (I) as defined above or their pharmaceutically acceptable salts can be prepared by a process comprising:

the reaction of the compound of formula (II)

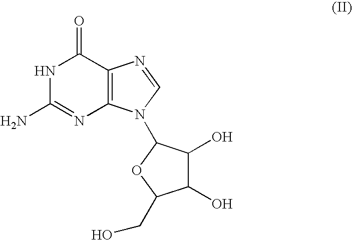

with a compound of formula (III)

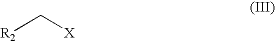

wherein

X is a suitable leaving group and R₂ is as defined above, to obtain a compound of formula (IV)

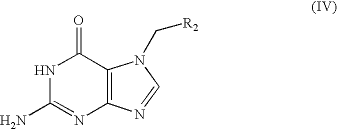

wherein R₂ is as defined above; and

the reaction of a compound of formula (IV) with a compound of formula (V)

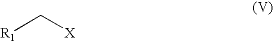

wherein

X is a suitable leaving group and R₁=R₂ or, more preferably, wherein R₁ is different from R₂ as defined above, so obtaining a compound of formula (I) and optionally salifying a compound of formula (I) as defined above, so obtaining a compound of formula (I) in the form of a pharmaceutically acceptable salt.

A compound of formula (IV) as defined above can be also represented by the following tautomeric formulae (Va), (Vb), (Vc) and (Vd):

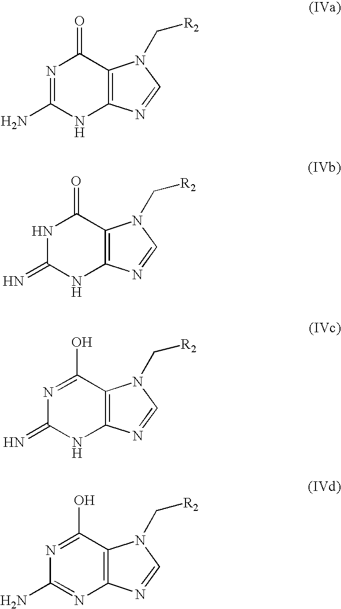

wherein R₂ is as defined above.

A suitable leaving group is, e.g., a halogen, preferably Cl, Br or I, tosylate, mesylate, trifluoroacetate and triflate.

The reaction between the compound of formula (II) as defined above and a compound of formula (III) as defined above can be performed in dimethylsulfoxide (DMSO) or DMF, at a temperature varying between about 20° C. and about 60° C., for a time of from about 1 hour to about 6 hours, as described, for example, in J. Org. Chem. 1986, 4180 or Synth. Comm. 1990, 2459.

The reaction between a compound of formula (IV), (IVa), (IVb), (IVc) or (IVd) and a compound of formula (V), may be carried out, for example, in a suitable organic solvent such as DMF, N,N-dimethylacetamide, dimethylsulfoxide, tetrahydrofuran, dioxane or dimethoxyethane, optionally in the presence of both an inorganic base such as sodium or potassium hydride, sodium, potassium or barium hydroxide, sodium or potassium carbonate, or an organic base such as, for instance, potassium terbutoxide, methyllithium, butyllithium, lithiumdiisopropylamine, lithium, sodium or potassium hexamethyldisilazide, at a temperature varying between room temperature and about 120° C., for a time of about 1 hour to about 15 hours, following, for example, literature methods as reported in Synth. Comm. 1990, 2459.

Examples of compounds prepared accordingly to this procedure are compounds 1-17.

Compounds of formula (II) and formula (III) are known compounds or can be prepared by known methods. For example, the compound of formula (II), which is the natural product guanosine and a compound of formula (III) can be prepared following methods well known in the art. For example, a compound of formula (III) can be prepared by halogenation reaction, with many methods known to people skilled in the art, starting from the corresponding alcohols that are commercially available or alternatively can be prepared, for example, from the corresponding commercial esters, by standard methods.

The compounds of the invention can be administered in a variety of dosage forms, e.g. orally, in the form of tablets, capsules, lozenges, liquid solutions or suspensions; rectally, in the form of suppositories; parenterally, e.g. intramuscularly, intravenously, intradermally or subcuteneously; or topically.

The dosage depends upon, for example, the compound of the invention employed, the age, weight, condition of the patient and administration route; specific dosage regimens may be fit to any particular subject on the basis of the individual need and the professional judgement of the person administering or supervising the administration of the aforesaid compounds.

For humans, therapy with the disclosed compounds includes doses of a pharmaceutical formulation comprising one or more of the compounds of the invention that are from about 0.001 to about 100 mg/kg. Preferably, the dosage is about 0.1 to 10 mg/kg. The dosages will vary in accordance with, for example, the condition of the patient and the type of disease being treated. A dosage can be administered once or can be divided into a number of smaller doses to be administered at varying intervals of time.

Pharmaceutical compositions containing, as an active ingredient, a compound of formula (I) or a pharmaceutically acceptable salt thereof in association with a pharmaceutically acceptable carrier and/or diluent, are also within the scope of the present invention.

These pharmaceutical compositions contain an amount of active ingredient, which is therapeutically effective to display antileukemic and/or antitumor activity. There may also be included as a part of the pharmaceutical compositions according to the invention, pharmaceutically acceptable binding agents and/or adjuvant materials. The active ingredients may aiso be mixed with other active principles, which do not impair the desired action and/or supplement the desired action.

The pharmaceutical compositions containing the compounds of the invention are usually prepared following conventional methods and may be administered in a pharmaceutically suitable form.

For example, the solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents, e.g. starches, arabic gums, gelatin, methylcellulose, microcrystalline cellulose, carboxymethylcellulose or polyvinyl pyrrolidone; diaggregating agents, e.g. a starch, alginic acid, alginates or sodium starch glycolate; effervescing mixtures; dyestuffs; sweetening agents, e.g. sucrose or saccharin; flavouring agents, e.g. peppermint, methylsalicylate or orange flavouring; wetting agents, such as lecithin, polysorbates, laurylsulphates; and, in general, non-toxic and pharmacologically inactive substances used in pharmaceutical formulations. When the dosage unit form is a capsule, it may contain, in addition to material of the above type, a liquid carrier such as, e.g., a fatty oil.

Said pharmaceutical preparations may be manufactured in known manner, for example, by means of mixing, granulating, tabletting, sugar-coating or film-coating processes. The liquid dispersions for oral administration may be, e.g. syrups, emulsions and suspensions.

The syrups may contain as carrier, for example, saccharose or saccharose with glycerine and/or mannitol and/or sorbitol; in particular, a syrup to be administered to diabetic patients can contain as carriers only products not metabolizable to glucose, or metabolizable in very small amount to glucose, for example sorbitol.

The suspensions and the emulsions may contain as carrier, for example, a natural gum, agar, sodium alginate, pectin, methylcellulose, carboxymethylcellulose, or polyvinyl alcohol.

The suspensions or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions may contain as carrier, for example, sterile water, or preferably they may be in the form of sterile, aqueous, isotonic saline solution. The solutions or suspensions for parenteral therapeutic administration may also contain antibacterial agents, such as benzyl alcohol or methyl parabens; antioxidants, such as ascorbic acid or sodium bisulphite; chelating agents, such as ethylenediaminetetraacetic acid; buffers, such as acetates, citrates or phosphates and agents for the adjustment of tonicity, such as sodium chloride or dextrose. The parenteral preparation can be enclosed in ampoules, disposable syringes or multiple dose vials made of glass or plastic.

The suppositories may contain together with the active compound a pharmaceutically acceptable carrier, e.g., coca-butter, polyethylene glycol, a polyoxyethylene sorbitan fatty acid ester surfactant or lecithin.

Compositions for topical application, such as, e.g., creams, lotions or pastes, may be, e.g., prepared by admixing the active ingredient with a conventional oleaginous or emulsifying excipient.

Biological Activity

The telomerase activity of the compounds of the invention has been evaluated using a Flash Plate-based assay. The method proved to be sensitive, accurate and able to reproducibly identify compounds that inhibit telomerase activity in a dose-dependent manner. Other methods for determining the inhibitory concentration of a compound of the invention against telomerase can be employed as will be apparent to a person skilled in the art based on the disclosure herein.

Briefly, the assay mixture is costituted by:

telomerase enzyme diluted in a buffer, the composition of which has been selected to maintain the enzyme activity stable along the duration of the assay.

dNTPs, deoxynucleotides 5′-triphosphate.

biotynilated oligo as primer.

increasing concentrations of test compounds/positive control.

After two hours of incubation at 370 degrees the telomeric repeats added are evaluated by hybridization in solution with a 3′-labeled short oligonucleotide probe.

The extent of hybridization is then quantitated by transferring the reaction mixture in a streptavidin-coated flash plate, where the binding between biotin and streptavidin occurs.

The telomerase activity is proportional to the radioactivity measured and the inhibitory activity of the compounds is evaluated as IC₅₀ using the Sigma Plot fit program.

With the above-described method, IC₅₀ values of the compounds of the present invention were determined.

The result relative to a representative compound of the invention is shown in Table 1 below.

TABLE 1

| Compound | IC₅₀ (μM) |
|---|---|
| 13 | 12.3 |

The datum reported in Table 1 clearly shows the activity of a representative compound according to the invention as telomerase inhibitor.

A human or animal body may thus be treated by a method, which comprises the administration thereto of a pharmaceutically effective amount of a compound of formula (I) or a salt thereof. The condition of the human or animal can thereby be improved.

The compounds of the invention can be administered either as single agents or, alternatively, in combination with one or more anti-cancer agent including, for example, topoisomerase inhibitors, antimetabolites, alkylating agents, antibiotics, antimicrotubule agents, hormonal agents, immunological agents, interferon-type agents, cyclooxygenase inhibitors (e.g. COX-2 inhibitors), metallomatrixprotease inhibitors, kinase inhibitors, tyrosine kinase inhibitors, anti-growth factor receptor agents, anti-HER agents, anti-EGFR agents, farnesyl transferase inhibitors, ras-raf signal transduction pathway inhibitors, cell cycle inhibitors, tubulin binding agents and anti-angiogenesi's agents.

Combinations of drugs are administered in an attempt to obtain a synergistic effect on most cancers, e.g., carcinomas, melanomas, sarcomas, lymphomas and leukemias and/or to reduce or eliminate emergence of drug-resistant cells and/or to reduce side effects to each drug.

It is therefore a still further aspect of the present invention a combined anti-cancer therapy which comprises administering a compound according to the invention with at least one other anti-cancer agent. The combined use of active substances provides improved therapeutic effect than employing the single agents alone.

Compounds of formula (I) may be combined with at least one other anti-cancer agent in a fixed pharmaceutical formulation or may be administered with at least one other anti-cancer agent in any desired order.

It is therefore a further object of the invention a product or kit comprising a compound of formula (I) or a pharmaceutically acceptable salt thereof of the invention and one or more anti-cancer agents for simultaneous, separate or sequential use in anticancer therapy.

Anti-cancer agents suitable for combination with the compounds of the present invention include, but are not limited to:

topoisomerase I inhibitors comprising, for example, epipodophyllotoxins such as, e.g. etoposide and teniposide; camptothecin and camptothecin derivatives including, e.g., irinotecan, SN-38, topotecan, 9-amino-camptothecin, 10,11-Methylenedioxy camptothecin and 9-nitro-camptothecin (rubitecan);

alkylating agents including nitrogen mustards such as, e.g., mechlorethamine, chlorambucil, melphalan, uracil mustard and estramustine; alkylsulfonates such as, e.g., busulfan improsulfan and piposulfan; oxazaphosphorines such as e.g., ifosfamide, cyclophosphamide, perfosfamide, and trophosphamide; platinum derivatives such as, e.g., oxaliplatin, carboplatin and cisplatin; nitrosoureas such as, e.g., carmustine, lomustine and streptozocin;

antimitotic agents including taxanes such as, e.g., paclitaxel and docetaxel; vinca alkaloids such as, e.g., vincristine, vinblastine, vinorelbine and vindesine; and novel microtubule agents such as, e.g., epothilone analogs, discodermolide analogs and eleutherobin analogs;

antimetabolites including purines such as, e.g., 6-mercaptopurine, thioguanine, azathioprine, allopurinol, cladribine, fludarabine, pentostatin, and 2-chloro adenosine; fluoropyrimidines such as, e.g., 5-FU, fluorodeoxyuridine, ftorafur, 5′-deoxyfluorouridine, UFT, S-1 and capecitabine; and pyrimidine nucleosides such as, e.g., deoxycytidine, cytosine arabinoside, 5-azacytosine, gemcitabine, and 5-azacytosine-arabinoside; antimetabolites (for example antifolates like methotrexate, fluoropyrimidines like 5-fluorouracil, purine and adenosine analogues, cytosine arabinoside;

hormones, hormonal analogues and hormonal antagonists including antiestrogens (for example tamoxifen, toremifen, raloxifene, droloxifene and iodoxyfene), progestogens (for example megestrol and acetate), aromatase inhibitors (for example anastrozole, letrazole, borazole and exemestane), antiprogestogens, antiandrogens (for example flutamide, nilutamide, bicalutamide and cyproterone acetate), LHRH agonists and antagonists (for example gosereline acetate and luprolide) and inhibitors of testosterone 5a-dihydroreductase (for example finasteride;

antitumor antibiotics including anthracyclines and anthracenediones such as, e.g., doxorubicin, daunorubicin, epirubicin, idarubicin and mitoxantrone;

farnesyltransferase inhibitors including, for example, SCH 44342, RPR 113228, BZA SB and PD 161956;

anti-invasion agents (for example metalloproteinase inhibitors such as, e.g., marimastat and inhibitors of urokinase plasminogen activator receptor functions);

inhibitors of growth factor (for example, EGF, FGF, platelet derived growth factor and hepatocyte growth factor) functions including growth factor antibodies, growth factor receptor antibodies, tyrosine kinase inhibitors and serine/threonine kinase inhibitors;

antiangiogenic agents such as, for example, linomide, inhibitors of integrin avβ3 function, angiostatin, razoxin, SU 5416, SU 6668, AGM 1470 (TNP-470), a synthetic analogue of fumagillin a naturally secreted product of the fungus Aspergillus fumigates fresenius, platelet factor 4 (endostatin), thalidomide, marimastat (BB-2516) and batimastat (BB-94);

cyclooxygenase (COX)-inhibitors, preferably COX-2 inhibitors such as, for example, celecoxib, parecoxib, rofecoxib, valecoxib and JTE 5222; and

cell cycle inhibitors such as, e.g., flavopyridols.

In a further aspect of this invention, a method is provided for treating a cancer comprising administering to a patient in need of such treatment a therapeutically effective amount of a compound of formula (I) as defined above and a therapeutically effective amount of at least another anti-cancer agent.

In a preferred aspect of the present invention, the combination of a compound of formula (I) as defined above with at least one antiangiogenesis agent is contemplated.

The terms “malignant neoplasm”, “cancer”, “tumor” and “solid tumor cancer” are used interchangeably herein to refer to the condition well known to those skilled in the art as the life-threatening disease commonly referred to simply as “cancer”. The term “cancer” as used herein, is meant a disease characterized by excessive, uncontrolled growth of abnormal cells, which invades and destroys other tissues and includes all human cancers such as carcinomas, sarcomas, leukemias and lymphomas. For example, the term “cancer” comprises prostate, breast, lung, colorectal, bladder, uterine, skin, kidney, pancreatic, ovarian, liver and stomach cancer.

By the term “chemotherapeutic agent” as used herein, is meant a chemical substance or drug used to treat a disease; the term is most often applied to such substances or drugs which are used primarily for the treatment of cancer.

By the term “treating” as used herein, is meant reversing, alleviating, ameliorating, limiting, inhibiting the progress of, or preventing the disorder or condition to which such term applies, or one or more symptoms of such disorder or condition. The term “treatment”¹ as used herein, refers to the act of treating as “treating” is defined immediately above.

By the term “method” as used herein, is meant manners, means, techniques and procedures for accomplishing a given task including, but not limited to, those manners, means, techniques and procedures either known to, or readily developed from known manners, means, techniques and procedures by, practitioners of the chemical, pharmacological, biological, biochemical and medical arts.

By the term “administered” or “administering” as used herein, is meant standard delivery methods, e.g. parenteral administration, including continuous infusion and intravenous, intramuscular and subcutaneous injections, and oral administration.

By the term “mammal” as used herein, is meant any of a class of warm-blooded higher vertebrates, that nourish their young with milk secreted by mammary glands, have the skin usually more or less covered with hair, and include humans.

By the term “pharmaceutically acceptable carrier” as used herein, is meant a carrier or diluent that does not cause significant irritation to an organism and does not abrogate the biological activity and properties of the administered compound.

By the term “excipient” as used herein, is meant an inert substance added to a pharmaceutical composition to further facilitate administration of a compound.

By the term “disease” as used herein, is meant a kind or instance of impairment of a living being that interferes with normal bodily function.

The following examples are provided for exemplification purposes only and are not intended to limit the scope of the invention described in broad terms above.

### EXAMPLE 1

### Compound 1

To a solution of guanosine hydrate (570 mg; 2 mmol) in anhydrous DMSO (3 mL) under argon 2-naphthylmethyl bromide (1.075 g; 4.8 mmol) is added and the reaction mixture stirred at room temperature for 4 h. Concentrated aq. HCl (1.5 mL) is added, the mixture is stirred for 0.5 h then poured into methanol (20 mL). The solution is concentrated and added of dichloromethane/methanol 2:1 (5 mL). The white precipitate is filtered and washed with dichloromethane. In this way 7-(2-naphthylmethyl) guanine hydrochloride (90% yield) is obtained and used for the second step.

To a suspension of 7-(2-naphthylmethyl) guanine hydrochloride (0.165 g, 0.5 mmol) in anhydrous DMF (4 mL), 60% NaH (0.050 g, 1.2 mmol) was added and the mixture was stirred at room temperature for 3 hours. A solution of 2-naphthylmethyl bromide (0.165 g, 0.75 mmol) in anhydrous DMF (1 mL) was added dropwise and the reaction mixture was stirred for 3 hours at room temperature. After solvent evaporation the crude reaction product was fractionated by flash chromatography (eluant dichloromethane/methanol 20:1) to yield 2-amino-1,7-bis(2-naphthylmethyl)-1,7-dihydro-6H-purin-6-one (0.09 g, 0.21 mmol, 31%), as a white solid.

¹H-NMR (400 Mhz, DMSOd₆), ppm: 5.35 (2H, s), 5.65 (2H, s), 6.75 (2H, a), 7.3-7.9 (14H, m), 8.2 (1H, s).

By analogous procedure the following compounds were prepared:

2-Amino-1-(3,4-dichlorobenzyl)-7-(2-naphthylmethyl)-1,7-dihydro-6H-purin-6-one (compound 2)

¹H-NMR (400 Mhz, DMSOd₆), ppm: 5.35 (2H, s), 5.6 (2H, s), 6.8 (2H, s), 7.3-7.9 (10H, m), 8.24 (1H, s).

2-Amino-7-(2-naphthylmethyl)-1-(3-phenoxybenzyl)-1,7-dihydro-6H-purin-6-one (compound 3)

¹H-NMR (400 Mhz, DMSOd₆), ppm: 5.25 (2H, s), 5.6 (2H, s), 6.8-8.0 (18H, m), 8.2 (1H, s).

2-Amino-7-(2-naphthylmethyl)-1-(2-quinolinylmethyl)-1,7-dihydro-6H-purin-6-one (compound 4)

¹H-NMR (400 Mhz, DMSO₆), ppm: 5.45 (2H, 9), 5.6 (2H, s), 6.75 (2H, s), 7.35 (1H, d, J=8.5 Hz), 7.4-7.95 (11H, m), 8.2 (1H, s), 8.35 (1H, d, J=8.5 Hz).

2-Amino-1-(2,1,3-benzoxadiazol-5-ylmethyl)-7-(2-naphthylmethyl)-1,7-dihydro-6H-purin-6-one (compound 5)

¹H-NMR (400 Mhz, DMSOd6), ppm: 5.35 (2H, s), 5.65 (2H, s), 6.8 (2H, s), 7.4-8.2 (10H, m), 8.25 (1H, s).

2-Amino-1-(1,3-benzodioxol-5-ylmethyl)-7-(2-naphthylmethyl)-1,7-dihydro-6H-purin-6-one (compound 6)

¹H-NMR (400 Mhz, DMSOd₆), ppm: 5.25 (2H, s), 5.6 (2H, s), 6.0 (2H, s), 6.85 (2H, s), 7.2-7.9 (10H, m), 8.2 (1H, s).

2-Amino-7-(2-naphthylmethyl)-1-(4-nitrobenzyl)-1,7-dihydro-6H-purin-6-one (compound 7)

¹H-NMR (400 Mhz, DMSOd₆), ppm: 5.2 (2H, s), 5.45 (2H, s), 6.7 (2H, s), 7.2-8.0 (11H, m), 8.2 (1H, s).

2-Amino-1-(3,4-difluorobenzyl)-7-(2-naphthylmethyl)-1,7-dihydro-6H-purin-6-one (compound 8)

¹H-NMR (400 Mhz, DMSOd₆), ppm: 5.15 (2H, s), 5.6 (2H, s), 6.7 (2H, s), 6.8-8.0 (10H, m), 8.2 (1H, s).

4-{[2-Amino-7-(2-naphthylmethyl)-6-oxo-6,7-dihydro-1H-purin-1-yl]methyl}benzonitrile (compound 9)

¹H-NMR (400 Mhz, DMSOd₆), ppm: 5.25 (2H, s), 5.6 (2H, s), 6.75 (2H, s), 7.2-8.0 (11H, m), 8.2 (1H, s).

2-Amino-1-[4-(1H-imidazol-1-yl)benzyl]-7-(2-naphthylmethyl)-1,7-dihydro-6H-purin-6-one (compound 10)

¹H-NMR (400 Mhz, DMSOd₆), ppm: 5.35 (2H, s), 5.6 (2H, s), 6.8 (2H, s), 7.05 (1H, s), 7.2-7.9 (12H, m), 8.2 (1H, B), 8.65 (1H, m).

2-{[2-Amino-7-(2-naphthylmethyl)-6-oxo-6,7-dihydro-1H-purin-1-yl]methyl}anthra-9,10-quinone (compound 11)

¹H-NMR (400 Mhz, DMSOd₆), ppm: 5.4 (2H, s), 5.65 (2H, s), 6.8. (2H, s), 7.35-8.25 (15H, m).

Methyl 4-{[2-amino-7-(2-naphthylmethyl)-6-oxo-6,7-dihydro-1H-purin-1-yl]methyl}benzoate (compound 12)

¹H-NMR (400 Mhz, DMSOd₆), ppm: 3.8 (3H, s), 5.25 (2H, s), 5.6 (2H, s), 6.7 (2H, s), 7.2-8.0 (11H, m), 8.2 (1H, s).

Methyl 4-({2-amino-7-[4-(methoxycarbonyl)benzyl]-6-oxo-6,7-dihydro-1H-purin-1-yl}methyl) benzoate (compound 13)

¹H-NMR (400 Mhz, DMSOd₆), ppm: 3.9 (6H, two s), 5.3 (2H, s), 5.6 (2H, s), 6.75 (2H, s), 7.3-7.9 (8H, m), 8.2 (1H, s).

Methyl 3-{[2-amino-7-(2-naphthylmethyl)-6-oxo-6,7-dihydro-1H-purin-1-yl]methyl}benzoate (compound 14)

¹H-NMR (400 Mhz, DMSOd₆), ppm: 3.8 (3H, s), 5.25 (2H, s), 5.6 (2H, s), 6.75 (2H, s), 7.4-8.0 (11H, m), 8.2 (1H, s).

Methyl 2-{[2-amino-7-(2-naphthylmethyl)-6-oxo-6,7-dihydro-1H-purin-1-yl]methyl}benzoate (compound 15)

¹H-NMR (400 Mhz, DMSOd₆), ppm: 3.85 (3H, s), 5.5 (2H, s), 5.6 (2H, s), 6.7 (2H, 8), 7.2-8.0 (10H, m), 8.2 (1H, s).

Methyl 4-{[2-amino-1-(2-naphthylmethyl)-6-oxo-1,6-dihydro-7H-purin-7-yl]methyl}benzoate (compound 16)

¹H-NMR (400 Mhz, DMSOd₆), ppm: 3.8 (3H, s), 5.3 (2H, s), 5.55 (2H, s), 6.7 (2H, s), 7.2-8.0 (11H, m), 8.15 (1H, s).

2-Amino-1-[(6-chloroimidazo[1,2-a]pyridin-2-yl)methyl]-7-(2-naphthylmethyl)-1,7-dihydro-6H-purin-6-one (compound 17)

¹H-NMR (400 Mhz, DMSOd₆), ppm: 5.25 (2H, s), 5.65 (2H, s), 6.85 (2H, s), 7.2-7.6 (5H, m), 7.7-8.0 (5H, m), 8.2 (1H, s), 8.75 (1H, m).

### EXAMPLE 2

### Intramuscular Injection of 50 mg/ml

A pharmaceutical injectable composition can be manufactured by dissolving 50 g of 2-amino-1,7-bis(2-naphthylmethyl)-1,7-dihydro-6H-purin-6-one (compound 1) in sterile propylene glycol (1000 ml) and sealed in 1-5 ml ampoules.

1. 1,7 disubstituted purines of formula (I) 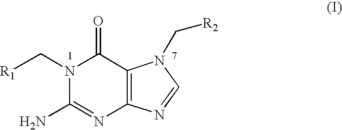
wherein
R1 and R2 represent each independently:
a) phenyl substituted by from 1 to 3 substituents chosen from halogen, cyano, nitro, amino, carboxyl, (C₁-C₆ alkoxy)carbonyl, phenyl and phenoxy;
b) a fused bicycle carbocyclic residue chosen from 1-naphthyl and 2-naphthyl;
c) imidazo[1,2-a]pyridin-2-yl optionally substituted with halogen;
d) a fused tricycle residue chosen from anthraquinonyl, phenothiazinyl, acridinyl and fluorenyl;
e) a fused benzoheterocycle residue chosen from benzodioxolyl, benzoxadiazolyl, quinolinyl, isoquinolinyl and quinazolinyl; or
f) a phenylheterocycle residue chosen from phenylimidazolyl, phenylpyrazolyl, phenyloxadiazolyl, phenylpyrrolyl, phenylfuranyl, phenylthiadiazolyl, phenyloxazolyl, phenylthiazolyl and phenyltetrazolyl;
and the pharmaceutically acceptable salts thereof.

2. A compound of formula (I) as claimed in claim 1 wherein R₁ and R₂ represent, each independently, phenyl substituted by from 1 to 3 substituents chosen from halogen, cyano, nitro, amino, carboxyl, (C₁-C₆ alkoxy)carbonyl, phenyl and phenoxy; 2-naphthyl; imidazo[1,2-a]pyridin-2-yl optionally substituted with chloro; anthraquinonyl; a fused benzoheterocyclicle residue chosen from benzodioxolyl, benzoxadiazolyl and quinolinyl; or phenylimidazolyl;
and the pharmaceutically acceptable salts thereof.

3. A compound selected from the group consisting of:
2-amino-1,7-bis(2-naphthylmethyl)-1,7-dihydro-6H-purin-6-one (compound 1);
2-amino-l-(3,4-dichlorobenzyl)-7-(2-naphthylmethyl)-1,7-dihydro-6H-purin-6-one (compound 2);
2-amino-7-(2-naphthylmethyl)-1-(3-phenoxybenzyl)-1,7-dihydro-6H-purin-6-one (compound 3);
2-amino-7-(2-naphthylmethyl)-1-(2-quinolinylmethyl)-1,7-dihydro-6H-purin-6-one (compound 4);
2-amino-1-(2,1,3-benzoxadiazol-5-ylmethyl)-7-(2-naphthylmethyl)-1,7-dihydro-6H-purin-6-one (compound 5);
2-amino-1-(1,3-benzodioxol-5-ylmethyl)-7-(2-naphthylmethyl)-1, 7-dihydro-6H-purin-6-one (compound 6);
2-amino-7-(2-naphthylmethyl)-1-(4-nitrobenzyl)-1,7-dihydro-6H-purin-6-one (compound 7);
2-amino-1-(3,4-difluorobenzyl)-7-(2-naphthylmethyl)-1,7-dihydro-6H-purin-6-one (compound 8);
4-{[2-amino-7-(2-naphthylmethyl)-6-oxo-6,7-dihydro-1H-purin-1-yl]methyl}benzonitrile (compound 9);
2-amino-1-[4-(1H-imidazol-1-yl)benzyl]-7-(2-naphthylmethyl)-1,7-dihydro-6H-purin-6-one (compound 10);
2-([2-amino-7-(2-naphthylmethyl)-6-oxo-6,7-dihydro-1H-purin-1-yl]methyl)anthra-9,10-quinone (compound 11);
methyl 4-{[2-amino-7-(2-naphthylmethyl)-6-oxo-6,7-dihydro-1H-purin-1-yl]methyl}benzoate (compound 12);
methyl 4-({2-amino-7-[4-(methoxycarbonyl)benzyl]-6-oxo-6,7-dihydro-1H-purin-1-yl}methyl) benzoate (compound 13);
methyl 3-{[2-amino-7-(2-naphthylmethyl)-6-oxo-6,7-dihydro-1H-purin-1-yl]methyl}benzoate (compound 14);
methyl 2-{[2-amino-7-(2-riaphthylmethyl)-6-oxo-6,7-dihydro-1H-purin-1-yl]methyl}benzoate (compound 15);
methyl 4-{[2-amino-1-(2-naphthylmethyl)-6-oxo-1,6-dihydro-7H-purin-7-yl]methyl}benzoate (compound 16);
2-amino-1-[(6-chloroimidaz6[1,2-a]pyridin-2-yl)methyl]-7-(2-naphthylmethyl)-1,7-dihydro-6H-purin-6-one (compound 17);
and the pharmaceutically acceptable salts thereof.

4. A method for inhibiting telomerase enzyme, which comprises contacting said enzyme with an effective amount of a compound having the formula(I) as defined in claim 1 or a pharmaceutically acceptable salt thereof.

5. A method for treating a telomerase-modulated disease, which comprises administering to a mammal a therapeutic effective amount of a compound having the formula (I) as defined in claim 1 or a pharmaceutically acceptable salt thereof.

6. A method for treating a cancer disease related to a deranged cancer cell growth mediated by telomerase enzyme activity, which comprises administering to a mammal a therapeutic effective amount of a compound having the formula (I) as defined in claim 1 or a pharmaceutically acceptable salt thereof.

7. A method for treating a cancer, which comprises administering to a mammal a therapeutic effective amount of a compound having the formula (I) as defined in claim 1 or a pharmaceutically acceptable salt thereof.

8. A pharmaceutical composition comprising a pharmaceutically acceptable carrier and/or diluent and, as an active principle, a compound of formula (I) as defined above or a pharmaceutically acceptable salt thereof.

9. A process for preparing a compound of formula (I) as defined in claim 1 comprising:
the reaction of the compound of formula (II) 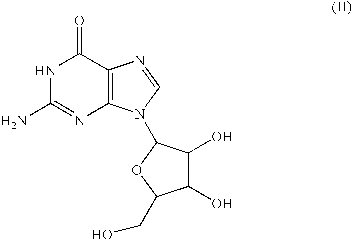
with a compound of formula (III) 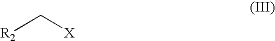
wherein
X is a suitable leaving group and R₂ is as defined above, to obtain a compound of formula (IV) 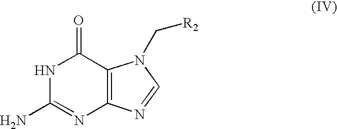
wherein R₂ is as defined above; and
the reaction of a compound of formula (IV) with a compound of formula (V) 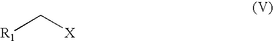
wherein
X is a suitable leaving group and R₁═R₂ or, more preferably, wherein R₁ is different from R₂ as defined above, so obtaining a compound of formula. (I) and optionally salifying a compound of formula (I) as defined above, so obtaining a compound of formula (I) in the form of a pharmaceutically acceptable salt.

10. A method for improving the therapeutic effect of a cancer therapy which comprises administering a therapeutically effective amount of a compound of formula (I) or a pharmaceutically acceptable salt thereof and at least another anticancer agent.

11. A kit comprising a compound of formula (I) or a pharmaceutically acceptable salt thereof and one or more anti-cancer agents for simultaneous, separate or sequential use in anticancer therapy.
